(19) United States
(12) Patent Application Publication
(10) Pub. No.: US 2020/0101217 A1
(43) Pub. Date: Apr. 2, 2020

(54) ENEMA DEVICE FOR WASHING LARGE INTESTINE

(71) Applicant: CHONBUK NATIONAL UNIVERSITY HOSPITAL, Jeonju-si (KR)

(72) Inventors: Jeong Hwan SEO, Jeonju-si (KR); Myoung Hwan KO, Jeonju-si (KR); Kap Soo HAN, Suwon-si (KR)

(73) Assignee: CHONBUK NATIONAL UNIVERSITY HOSPITAL, Jeonju-si (KR)

(21) Appl. No.: 16/613,428

(22) PCT Filed: May 15, 2018
(86) PCT No.: PCT/KR2018/005559

§ 371 Date: Nov. 14, 2019

(30) Foreign Application Priority Data
May 15, 2017 (KR) 10-2017-0059687

(51) Int. Cl. A61M 3/02; A61M 3/06; A61M 39/22

(52) U.S. Cl. CPC A61M 3/0295; A61M 2025/1061; A61M 39/223; A61M 3/06

(57) ABSTRACT

An enema device for washing a large intestine that can supply water to a large intestine to easily wash and defecate the large intestine. The enema device for washing a large intestine includes a nozzle part inserted into an anal passage and having an ejection hole for ejecting water at one end thereof; a balloon part coupled to an outer peripheral surface of the nozzle part to be expanded; an air pump configured to supply air to the balloon part; a switching valve, to which the air pump is connected; a first water hose, an air hose, and a second water hose; the nozzle part has a discharge hole through which air supplied from the air hose is discharged to the balloon part.

### CROSS-REFERENCES TO THE RELATED APPLICATIONS

This application is the national phase entry of International Application No. PCT/KR2018/005559, filed on May 15, 2018, which is based upon and claims priority to Korean Patent Application No. 10-2017-0059687, filed on May 15, 2017, the entire contents of which are incorporated herein by reference.

### TECHNICAL FIELD

The present invention relates to an enema device for washing a large intestine, and more particularly to an enema device for washing a large intestine that can supply water to a large intestine to easily wash and defecate the large intestine.

### BACKGROUND

Handicapped people or people having troubles in moving have difficulty or no ability in discharging stool to the outside of their bodies according to occasions.

In particular, the large intestines of the lower half body paralyzed people hardly move and there is a difficulty in defecation.

If the states continue, malicious constipations that require medical treatments occur.

General treatment methods against the problems include injecting an enema agent into an anal passage or extracting hardened stool with a physical method, and in severe cases, an artificial anal passage is attached through a surgery.

Various tools have been recently developed to solve the problems, but the tools are inconvenient for handicapped people to use.

### SUMMARY

The present invention has been made in an effort to solve the above-mentioned problems, and provides an enema device for washing a large intestine, which is connected to a bidet to automatically supply water to a large intestine so that handicapped people also can easily use the enema device.

An enema device for washing a large intestine according to an aspect of the present invention may include: a nozzle part inserted into an anal passage and having an ejection hole for ejecting water at one end thereof; a balloon part coupled to an outer peripheral surface of the nozzle part to be expanded; an air pump configured to supply air to the balloon part; a switching valve, to which the air pump is connected; a first water hose, one end of which is connected to an opposite end of the nozzle part and an opposite end of which is connected to the switching valve such that water flows through the first water hose; an air hose, one end of which is connected to the opposite end of the nozzle part and an opposite end of which is connected to the switching valve such that air flows through the air hose; and a second water hose, one end of which is connected to the switching valve and an opposite end of which is connected to a water supply hose of a bidet such that water flows through the second water hose, wherein the nozzle part has a discharge hole, through which air supplied from the air hose is discharged to the balloon part, and wherein the switching valve opens and closes connection of the air pump and the air hose to control supply of air from the air pump to the balloon part, and opens and closes connection of the first water hose and the second water hose to control supply of water from the water supply hose of the bidet to the nozzle part.

The switching valve may have a water input port, an air input port, a water output port, and an air output port, the second water hose may be connected to the water input port, the air pump is connected to the air input port, the first water hose is connected to the water output port, and the air hose is connected to the air output port, and the switching valve may open and close connection of the water input port and the water output port, and may open and close connection of the air input port and the air output port.

The switching valve may include: a housing having the water input port, the air input port, the water output port, and the air output port; a liquid passage formed in the interior of the housing and connecting the water input port and the water output port; an air passage formed in the interior of the housing and connecting the air input port and the air output port; a liquid control valve mounted to the housing and configured to open and close the liquid passage; and an air control valve mounted on the housing and configured to open and close the air passage.

The liquid control valve and the air control valve may be rotatably mounted on the housing, and the liquid control valve may control the pressure of water flowing through the liquid passage through rotation thereof.

Each of the liquid control valve and the air control valve may include: a knob exposed to the outside of the housing and rotated by an external force; and a rotary body disposed in the interior of the liquid passage and the air passage, having a through-hole, and rotated together with the knob, and the through-hole formed in the rotary body may be rotated to open and close the corresponding one of the liquid passage and the air passage when the knob is rotated.

The second water hose may be detachably coupled to the water supply hose of the bidet.

The enema device may further include: a pressure sensor mounted on the nozzle part and configured to measure the pressure of a large intestine; and a control unit configured to control the amount of water supplied through the water supply hose of the bidet, and the control unit interrupts flow of the water supplied through the water supply hose of the bidet if the pressure of the large intestine measured by the pressure sensor becomes a preset pressure.

The enema device for washing a large intestine of the present invention has the following effects.

A general person can recognize washing of a large intestine by supplying water to the large intestine, and a handicapped person of a lower body can easily be defecated by supplying water to the large intestine.

In particular, according to the present invention, because the water is supplied though the bidet, the temperature and the water pressure of the water may be easily controlled, and handicapped people can easily use the enema device because it is not necessary for the user to perform pumping to supply water, and the enema can be easily carried and preserved because it has not water bag for supplying water.

### BRIEF DESCRIPTION OF THE DRAWINGS

The above and other objects, features, and advantages of the present invention will be more apparent from the following detailed description taken in conjunction with the accompanying drawings, in which:

FIG. 1 shows a diagram of an enema device for washing a large intestine according to an embodiment of the present invention;

FIGS. 2A and 2B are views of a structure of a switching valve of the enema device for washing a large intestine according to the embodiment of the present invention;

FIGS. 3A and 3B are views of an operational state of the switching valve of the enema device for washing a large intestine according to the embodiment of the present invention;

FIG. 4 shows a view of a state in which the enema device for washing a large intestine is inserted into an anal passage according to the embodiment of the present invention;

FIG. 5 shows a view of a state in which a balloon is expanded in FIG. 4;

FIG. 6 shows a view of a state in which water is ejected through a nozzle part in FIG. 5; and

FIG. 7 shows a view of a state in which the nozzle part is removed in FIG. 6.

### DETAILED DESCRIPTION OF THE EMBODIMENTS

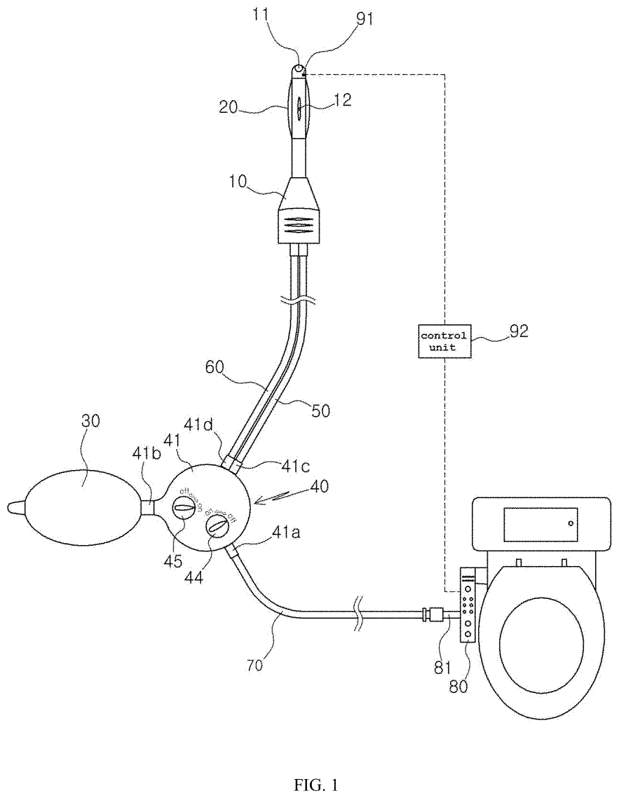

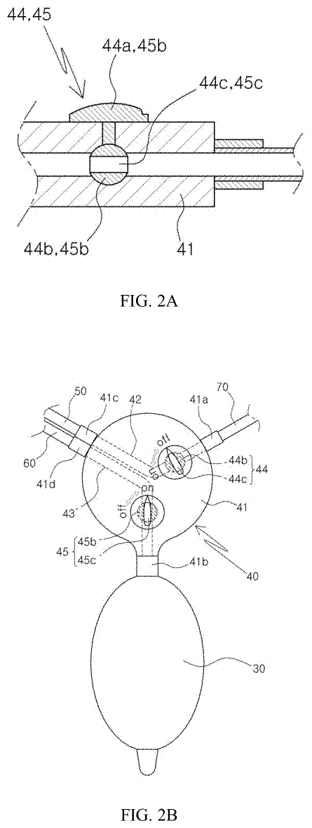

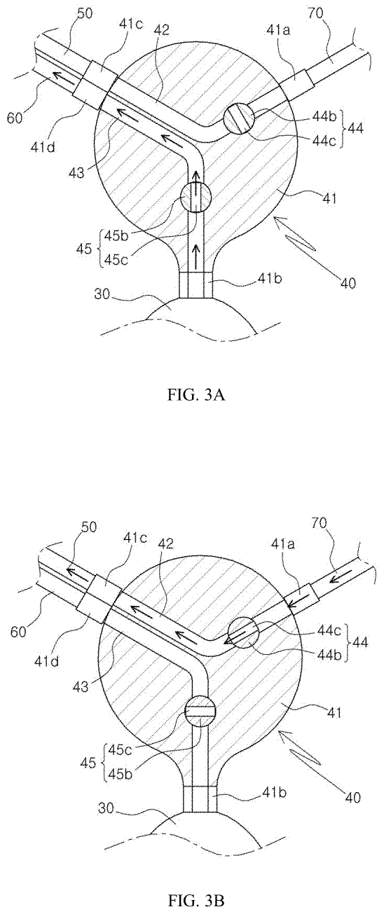

FIG. 1 is a diagram of an enema device for washing a large intestine according to an embodiment of the present invention. FIGS. 2A and 2B are views of a structure of a switching valve of the enema device for washing a large intestine according to the embodiment of the present invention. FIGS. 3A and 3B are views of an operational state of the switching valve of the enema device for washing a large intestine according to the embodiment of the present invention.

An enema device for washing a large intestine of the present invention includes a nozzle part 10, a balloon part 20, an air pump 30, a switching valve 40, a first water hose 50, an air hose 60, and a second water hose 70.

The nozzle part 10 is a part that is inserted into an anal passage, and an ejection hole 11 for ejecting water is formed at one end of the nozzle part 10.

The balloon part 20 is coupled to an outer peripheral surface of the nozzle part 10, and is expanded by air supplied from the outside.

To achieve this, the nozzle part 10 has a discharge hole 12, through which the air supplied from the air hose 60 is discharged to the balloon part 20, and the air supplied from the outside is introduced into the balloon part 20 through a passage formed in the interior of the nozzle part 10 and the discharge hole 12 to expand the balloon part 20.

The air pump 30 supplies air to the balloon part 20 and may include an electric motor pump, but it is preferable that the air pump 30 includes a manual rubber pump for portability and the like.

The switching valve 40 controls supply of air to the balloon part 20, and controls supply of water to the nozzle part 10.

The air pump 30, the first water hose 50, the air hose 60, and the second water hose 70 are connected to the switching valve 40 that controls supply of air and water.

One end of the first water hose 50 is connected to an opposite end of the nozzle part 10, and an opposite end of the first water hose 50 is connected to the switching valve 40 such that water flows through the first water hose 50.

One end of the air hose 60 is connected to the opposite end of the nozzle part 10, and an opposite end of the air hose 60 is connected to the switching valve 40 such that air flows through the air hose 50.

Then, the one end of the air hose 60 connected to the opposite end of the nozzle part 10 communicates with the discharge hole 12 formed in the nozzle part 10, and the air is supplied to the balloon part 20 through the air hose 60.

One end of the second water hose 70 is connected to the switching valve 40, and an opposite end of the second water hose 70 is connected to a water supply hose 81 of a bidet 80 such that water flows through the second water hose 70.

Then, the second water hose 70 is detachably coupled to the water supply hose 81 of the bidet 80 through an adapter or the like.

The switching valve 40 opens and closes connection of the air pump 30 and the air hose 60 to control supply of air from the air pump 30 to the balloon part 20, and opens and closes connection of the first water hose 50 and the second water hose 70 to supply water from the water supply hose 81 of the bidet 80 to the nozzle part 10.

To achieve this, the switching valve 40 includes a housing 41, a liquid passage 42, an air passage 43, a liquid control valve 44, and an air control valve 45.

The housing 41 has a water input port 41a, an air input port 41b, a water output port 41c, and an air output port 41d.

The second water hose 70 is connected to the water input port 41a, and the air pump 30 is connected to the air input port 41b.

The first water hose 50 is connected to the water output port 41c, and the air hose 60 is connected to the air output port 41d.

The liquid passage 42 is formed in the interior of the housing 41, and connects the water input port 41a and the water output port 41c related to the flow of water.

The air passage 43 is formed in the interior of the housing 41, and connects the air input port 41b and the air output port 41d related to the flow of air.

The liquid control valve 44 is mounted on the housing 41 to open and close the liquid passage 42.

The air control valve 45 is mounted on the housing 41 to open and close the air passage 43.

The liquid control valve 44 and the air control valve 45 are rotatably mounted on the housing 41, and the liquid control valve 44 controls the pressure of water flowing through the liquid passage 42.

The switching valve 40, through the air control valve 45 and the liquid control valve 44, opens and closes connection of the water input port 41a and the water output port 41c to control supply of water supplied to the nozzle part 10, and opens and closes connection of the air input port 41b and the air output port 41d to control supply of air supplied to the balloon part 20.

Each of the liquid control valve 44 and the air control valve 45 includes a knob 44a and 45a exposed to the outside of the housing 41 and rotated by an external force, and a rotary body 44b and 45b disposed in the interior of the corresponding one of the liquid passage 42 and the air passage 43, having a through-hole 44c and 45c, and a rotary body 44b and 45b rotated together with the knob 44a and 45a.

When the knob 44a and 45a is rotated, the through-hole 44c and 45c formed in the rotary body 44b and 45b is rotated to open and close the corresponding one of the liquid passage 42 and the air passage 43.

The enema device of the present invention uses the bidet 80 installed conventionally for supply of water, and uses the air pump 30 for supply of air.

Because water is supplied by using the bidet 80, the temperature, the water pressure, and the like of the water may be controlled by using the function of the bidet 80, and in particular, the water pressure of the water may be controlled once again by using the liquid control valve 44 so that the ejection of the water of high pressure may be controlled.

Meanwhile, the present invention may further include a pressure sensor 91 mounted on the nozzle part 10 and configured to measure the pressure of the large intestine, and a control unit 92 configured to control the amount of water supplied through the water supply hose 81 of the bidet 80.

The pressure sensor 91 is mounted on an end of the nozzle part 10, and measure the pressure of the large intestine, to which water is supplied.

The control unit 92 interrupts flow of the water supplied through the water supply hose 81 of the bidet 80 if the pressure of the large intestine measured by the pressure sensor 91 becomes a preset pressure.

Then, units for interrupting the flow of the water supplied through the water supply hose 81 of the bidet 80 may include a bidet 80 itself, the liquid control valve 44, or a separate valve.

The control unit 92 is connected to the bidet 80, and the water supplied from the bidet 80 may be controlled by controlling the bidet 80.

Further, the liquid control valve 44 may be motor-driven, and the control unit 92 may be connected to the liquid control valve 44 to control the liquid control valve 44.

Further, a separate solenoid valve may be mounted such that the control unit 92 controls the solenoid valve.

In this way, by controlling the amount of water supplied to the large intestine through the pressure sensor 91 and the control unit 92, only a predetermined amount of water may be safely supplied while a too large amount of water is not supplied to the large intestine.

Hereinafter, an operational process of the present invention including the above configuration will be described.

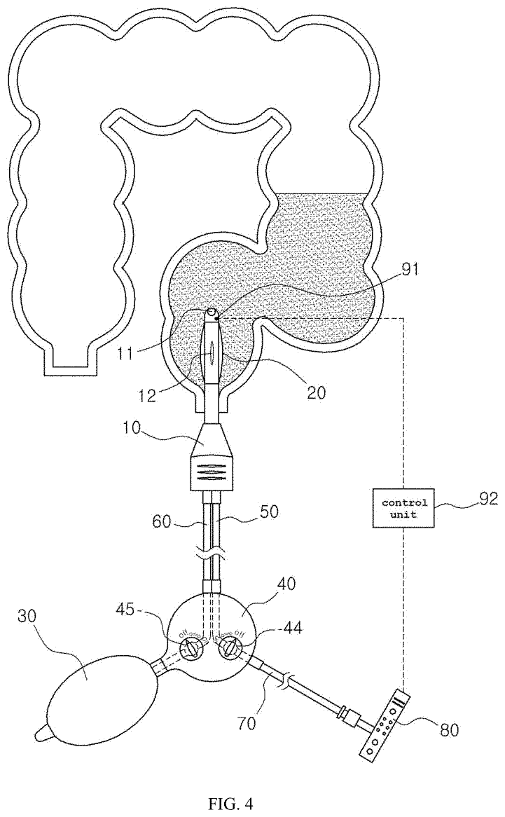

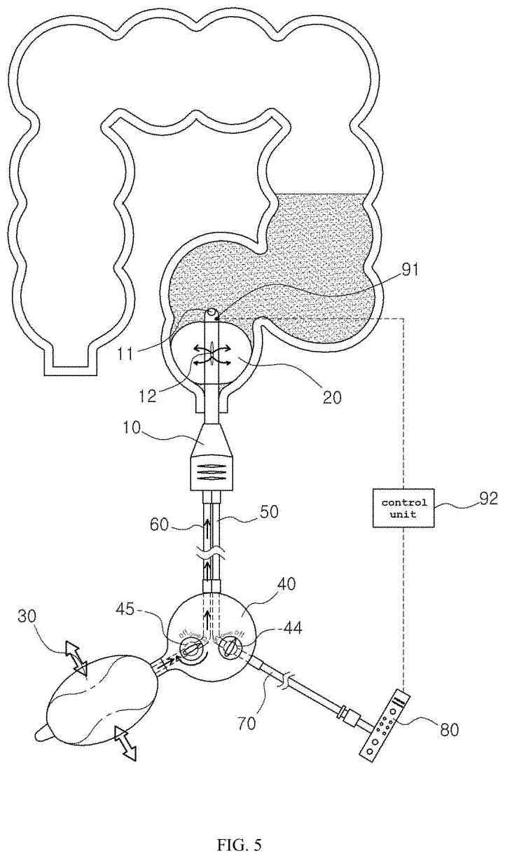

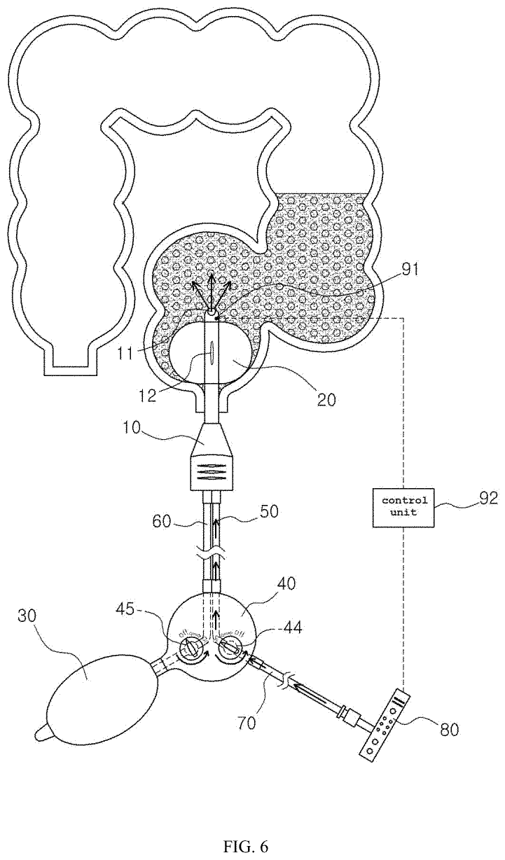

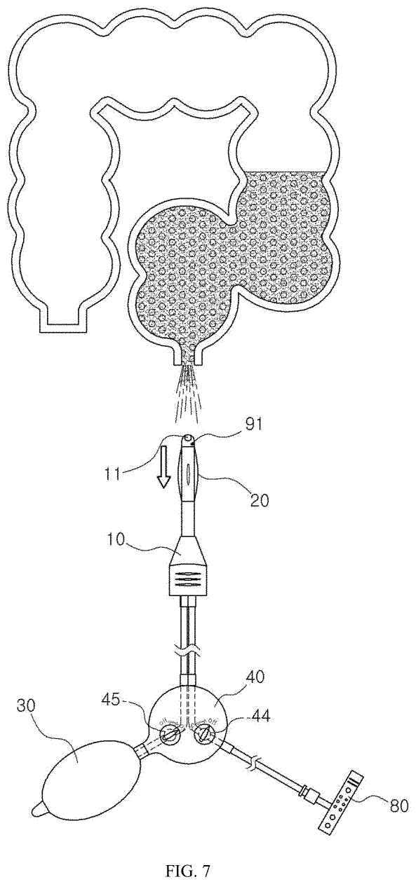

FIG. 4 is a view of a state in which the enema device for washing a large intestine is inserted into an anal passage according to the embodiment of the present invention. FIG. 5 is a view of a state in which a balloon is expanded in FIG. 4. FIG. 6 is a view of a state in which water is ejected through a nozzle part 10 in FIG. 5. FIG. 7 is a view of a state in which the nozzle part 10 is removed in FIG. 6.

First, the second water hose 70 is coupled to the water supply hose 81 of the bidet 80.

Further, as illustrated in FIG. 4, the nozzle part 10 is inserted into an anal passage such that the balloon part 20 is inserted into the large intestine.

Then, both the air passage 43 and the liquid passage 42 are closed.

Thereafter, the air passage 43 is opened by rotating the air control valve 45, and the air is supplied by using the air pump 30.

Then, the liquid passage 42 maintains the closed state.

Then, as illustrated in FIG. 5, the air supplied from the air pump 30 is supplied to the balloon part 20 through the air input port 41b, the air passage 43, the air output port 41d, the air hose 60, and the discharge hole 12.

The balloon part 20 is expanded as the air is supplied, and is attached to an inner wall of the large intestine.

Thereafter, the air control valve 45 is rotated to close the air passage 43, and the liquid control valve 44 is rotated to open the liquid passage 42.

The water supplied from the bidet 80 as the liquid passage 42 is opened, as illustrated in FIG. 6, flows through the second water hose 70, the liquid input port, the liquid passage 42, the liquid output port, the first water hose 50, and the nozzle part 10, and is ejected from the ejection hole 11 to the interior of the large intestine.

Then, the user may control the amount and the pressure of the water flowing through the liquid passage 42 by controlling the rotation degree of the knob 44a and 45a of the liquid control valve 44 and thus controlling the opening angle of the through-hole 44c and 45c formed in the rotary body 44b and 45b.

As water is supplied to the large intestine, the large intestine is washed, moisture is supplied to stool, and the large intestine is moved.

As water is supplied to the large intestine, the pressure of the large intestine measured by the pressure sensor 91 gradually increases.

The control unit 92 interrupts the water supplied through the bidet 80 such that the water is not further supplied to the large intestine if the pressure of the large intestine measured by the pressure sensor 91 reaches a preset value.

If a preset amount of water is supplied to the large intestine in advance, the air control valve 45 is opened such that the air of the balloon part 20 is discharged to the outside to be contracted, and the nozzle part 10 is extracted from the anal passage at the same time.

Then, as illustrated in FIG. 7, the stool may be easily discharged while the water supplied to the large intestine is discharged through the anal passage at the same time.

Through this, a general person can recognize washing of a large intestine, and a handicapped person of a lower body can easily be defecated.

In particular, according to the present invention, because the water is supplied though the bidet 80, the temperature and the water pressure of the water may be easily controlled, and the uncomfortable feeling of the user can be reduced because it is not necessary for the user to perform pumping to supply water, and the enema can be easily carried and preserved because it does not have water bag for supplying water.

The enema device for washing a large intestine of the present invention is not limited to the above-described embodiments, and may be variously modified within a range that is allowed by the technical sprit of the present invention.

### INDUSTRIAL APPLICABILITY

The present invention may be applied to an enema device for washing a large intestine, and thus can be industrially applicable.

What is claimed is:

1. An enema device for washing a large intestine, comprising:
A nozzle part configured to be inserted into an anal passage and having an ejection hole for ejecting water at one end thereof;
a balloon part coupled to an outer peripheral surface of the nozzle part to be expanded;
an air pump configured to supply air to the balloon part;
a switching valve, wherein the air pump is connected to the switching valve;
a first water hose, one end of the first water hose is connected to an opposite end of the nozzle part and an opposite end of the first water hose is connected to the switching valve such that water flows through the first water hose;
an air hose, one end of the air hose is connected to the opposite end of the nozzle part and an opposite end of the air hose is connected to the switching valve such that air flows through the air hose; and
a second water hose, one end of the second water hose is connected to the switching valve and an opposite end of the second water hose is connected to a water supply hose of a bidet such that water flows through the second water hose,
wherein the nozzle part has a discharge hole, the air supplied from the air hose is discharged to the balloon part through the discharge hole, and
wherein the switching valve opens and closes a connection of the air pump and the air hose to control supply of air from the air pump to the balloon part, and opens and closes a connection of the first water hose and the second water hose to control supply of water from the water supply hose of the bidet to the nozzle part.

2. The enema device of claim 1, wherein the switching valve has a water input port, an air input port, a water output port, and an air output port,
wherein the second water hose is connected to the water input port, the air pump is connected to the air input port, the first water hose is connected to the water output port, and the air hose is connected to the air output port, and
wherein the switching valve opens and closes connection of the water input port and the water output port, and opens and closes connection of the air input port and the air output port.

3. The enema device of claim 2, wherein the switching valve comprises:
a housing having the water input port, the air input port, the water output port, and the air output port;
a liquid passage formed in an interior of the housing and connecting the water input port and the water output port;
an air passage formed in the interior of the housing and connecting the air input port and the air output port;
a liquid control valve mounted to the housing and configured to open and close the liquid passage; and
an air control valve mounted on the housing and configured to open and close the air passage.

4. The enema device of claim 3, wherein the liquid control valve and the air control valve are rotatably mounted on the housing, and
wherein the liquid control valve controls a pressure of water flowing through the liquid passage through a rotation thereof.

5. The enema device of claim 4, wherein each of the liquid control valve and the air control valve comprises:
a knob exposed to an outside of the housing and is configured to be rotated by an external force; and
a rotary body disposed in an interior of the liquid passage and the air passage, having a through-hole, and is configured to rotate together with the knob, and
wherein the through-hole formed in the rotary body and the rotary body is rotated to open and close a corresponding one of the liquid passage and the air passage when the knob is rotated.

6. The enema device of claim 1, wherein the second water hose is detachably coupled to the water supply hose of the bidet.

7. The enema device of claim 1, further comprising:
a pressure sensor mounted on the nozzle part and configured to measure a pressure of a large intestine; and
a control unit configured to control an amount of water supplied through the water supply hose of the bidet,
wherein the control unit interrupts flow of the water supplied through the water supply hose of the bidet if the pressure of the large intestine measured by the pressure sensor becomes a preset pressure.
